(19) United States
(12) Patent Application Publication
(10) Pub. No.: US 2019/0110662 A1
(43) Pub. Date: Apr. 18, 2019

(54) ENDOSCOPE

(71) Applicant: Olympus Corporation, Tokyo (JP)

(72) Inventors: Tatsuya Obara, Tokyo (JP); Takanori Watanabe, Tokyo (JP); Jin Ito, Tokyo (JP)

(73) Assignee: Olympus Corporation, Tokyo (JP)

(21) Appl. No.: 16/218,647

(22) Filed: Dec. 13, 2018

Related U.S. Application Data
(63) Continuation of application No. PCT/JP2017/020748, filed on Jun. 5, 2017.

(30) Foreign Application Priority Data
Jun. 14, 2016 (JP) 2016-117731

(51) Int. Cl. A61B 1/00; A61B 1/015; A61B 1/05

(52) U.S. Cl. CPC A61B 1/00094; A61B 1/00181; A61B 1/00009; A61B 1/015; A61B 1/05; A61B 1/00096

(57) ABSTRACT

An endoscope comprises an insertion portion having a distal-end surface configured to be inserted into a body. The insertion portion includes a suction port being formed in the distal-end surface for drawing in liquid from the body. A protrusive portion having a passageway formed thereto. The protrusive portion is protruded from the distal-end surface. The passageway is formed in the protrusive portion to remove the liquid positioned in facing relationship with respect to the distal-end surface and opposite of the suction port across the protrusive portion toward the suction port.

### CROSS-REFERENCE TO RELATED APPLICATIONS

This application is a continuation application of PCT Application No. PCT/JP2017/020748 filed on Jun. 5, 2017, which in turn claim priority to the Japanese Patent Application No. 2016-117731 filed on Jun. 14, 2016 in Japan which is hereby incorporated by reference in its entirety.

### TECHNICAL FIELD

The technology disclosed herein relates to an endoscope having a suction port and a protrusive portion. The suction port is defined in a distal-end surface of an insertion portion to be inserted into a body for drawing in a liquid from the body. The protrusive portion protrudes in a columnar shape from the distal-end surface.

### DESCRIPTION OF THE RELATED ART

In recent years, endoscopes have widely been used in the medical field and the industrial field. An endoscope is able to observe the inside of a body by inserting a slender insertion portion into the body.

Endoscopes include a direct-viewing endoscope and a side-viewing endoscope. The direct-viewing endoscope has an observation lens and an illumination lens on a distal-end portion on a distal-end side of an insertion portion thereof. The side-viewing endoscope has an observation lens and an illumination lens on part of an outer circumferential surface of a distal-end portion of an insertion portion thereof.

In recent years, to widen an observation range inside the body, there is another endoscope that is capable of simultaneously observing not only a region along a longitudinal direction of an insertion portion thereof, but also the field of vision of a region including a direction transverse to the longitudinal direction, as disclosed in Japanese Patent No. 5715308.

A specific example of the region along the longitudinal direction is a region forward of the distal-end surface of the insertion portion. A specific example of the region including the direction transverse to the longitudinal direction is a circumferential region positioned around an outer circumferential surface of the distal-end portion that lies substantially perpendicularly to the longitudinal direction. The endoscope disclosed in Japanese Patent No. 5715308 has a protrusive portion including a first protrusive part and a second protrusive part that protrude in a columnar shape forwardly from a distal-end surface of a distal-end portion of an insertion portion of the endoscope.

The first protrusive part houses therein a forward observation lens acting as a forward subject image acquiring section in facing relation to a distal-end surface of the first protrusive part. The first protrusive part also houses therein a circumferential observation lens disposed rearwardly of the forward observation lens and acting as a side subject image acquiring section along an outer circumferential surface of the first protrusive part. The distal-end portion houses therein a lens group and an image capturing section. The lens group is positioned rearwardly of the circumferential observation lens. The image capturing section such as a charge-coupled device (CCD) or the like is disposed at the focused position of the lens group.

A subject that is positioned forwardly of the distal-end surface of the first protrusive part is captured as a forward subject image by the forward observation lens. Light applied to the forward observation lens passes through the circumferential observation lens and is focused onto the image capturing section by the rearward lens group.

A subject that is positioned in a circumferential direction of the first protrusive part is captured as a side subject image by the circumferential observation lens. Light applied to the circumferential observation lens is reflected a plurality of times in the circumferential observation lens and thereafter focused onto the image capturing section by the rearward lens group. A display section displays the forward subject image in a substantially circular shape and displays the side subject image in a substantially C shape around the outer circumference of the forward subject image, except for a region blocked by the second protrusive part. This arrangement allows the user to observe not only a forward field of vision, but also a circumferential field of vision at the same time on the display section. In other words, the user is able to observe a wide range inside the body.

Furthermore, the second protrusive part has an illumination lens and a nozzle on the distal-end surface of the second protrusive part. The illumination lens illuminates a subject positioned forwardly. The nozzle supplies a fluid to a forward observation lens. The second protrusive part also has on an outer circumferential surface thereof a nozzle for supplying a fluid to a circumferential observation lens.

Moreover, a suction channel for drawing in dirt and a liquid such as blood or the like from the body has a suction port that is open in the distal-end surface of the distal-end portion. When a liquid from the body is drawn using the suction port of the endoscope disclosed in Japanese Patent No. 5715308, if the liquid is facing the suction port in the distal-end surface, then the liquid can easily be drawn in from the suction port. However, if the liquid is positioned opposite from the suction port across the protrusive portion on the distal-end surface, then the liquid is obstructed by the protrusive portion, particularly the second protrusive part, and cannot be drawn in from the suction port.

If the liquid is blood or the like, then the liquid always needs to be removed under suction as it may make it difficult to observe a lesion when applied to the lesion.

In such a case, it has heretofore been customary for the user to change the position of the distal-end surface of the distal-end portion in the body so as to bring the suction port into facing relationship with the liquid so as to draw the liquid from the suction port.

According to the above practice, however, the forward observation lens or the circumferential observation lens in the body may possibly be shifted, displacing the observational field of vision off the area of interest in the body.

A suction port may be formed in the distal-end surface or outer circumferential surface of the protrusive portion for drawing in the liquid without being obstructed by the protrusive portion while keeping the observational field of vision fixed.

However, since the area of the distal-end surface or outer circumferential surface of the protrusive portion is very small compared with the distal-end surface of the distal-end portion, it is difficult for the suction port to have the same opening area as the suction port that is open in the distal-end surface. Consequently, the amount of the liquid drawn is reduced. In addition, the user tends to feel awkward and uneasy due to a different process of drawing in the liquid because the position of the opening of the suction port is different from the corresponding position available hereinbefore.

Accordingly, there is a need for an endoscope capable of observing not only a forward field of vision, but also a circumferential field of vision at the same time to have an arrangement for drawing in a liquid from a body without moving an observational field of vision. The liquid is positioned opposite a suction port across a protrusive portion from the suction port without being obstructed by the protrusive portion that protrudes from a distal-end surface.

### BRIEF SUMMARY OF EMBODIMENTS

The disclosed technology is directed to an endoscope incorporating an arrangement for drawing in liquid from a body via a suction port without moving an observational field of vision. The suction port is located in a distal-end surface of a distal-end portion of an insertion portion. The liquid is positioned opposite the suction port across a protrusive portion that protrudes from the distal-end surface of the distal-end portion

The disclosed technology is directed to an endoscope comprises an insertion portion having a distal-end surface configured to be inserted into a body. The insertion portion includes a suction port being formed in the distal-end surface for drawing in liquid from the body. A protrusive portion having a passageway formed thereto. The protrusive portion is protruded from the distal-end surface. The passageway is formed in the protrusive portion to remove the liquid positioned in facing relationship with respect to the distal-end surface and opposite of the suction port across the protrusive portion toward the suction port.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a perspective view schematically illustrating an example of an endoscopic apparatus including an endoscope and a peripheral device according to a first embodiment.

FIG. 2 is a fragmentary perspective view illustrating at an enlarged scale a distal-end portion depicted in FIG. 1.

FIG. 3 is a plan view of the distal-end portion depicted in FIG. 2, as viewed from the direction III in FIG. 2.

FIG. 4 is a view illustrating a forward subject image and a side subject image displayed on a monitor depicted in FIG. 1.

FIG. 5 is a view schematically illustrating the manner in which a liquid in a body is drawn from a suction port into a suction channel through a passageway depicted in FIG. 2.

FIG. 6 is a fragmentary perspective view of a distal-end portion according to a modification in which the passageway depicted in FIG. 2 is defined as a groove.

FIG. 7 is a fragmentary perspective view of a distal-end portion according to a modification in which the passageway depicted in FIG. 2 is defined as a space created by a tapered surface on a second protrusive part.

FIG. 8 is a fragmentary perspective view schematically illustrating at an enlarged scale a distal-end portion of an insertion portion of an endoscope according to a second embodiment.

FIG. 9 is a plan view of the distal-end portion depicted in FIG. 8, as viewed from the direction IX in FIG. 8.

FIG. 10 is a plan view illustrating a modification in which the distal-end portion depicted in FIG. 8 has a groove defined in a distal-end surface thereof and providing fluid communication between a suction port and the opening of a forward water delivery channel.

FIG. 11 is a fragmentary perspective view illustrating a modification in which a plurality of suction holes are defined in a cover that covers an outer circumferential surface of the distal-end portion depicted in FIG. 8.

FIG. 12 is a cross-sectional view of a distal-end portion and a cover, taken along line XII-XII of FIG. 11.

FIG. 13 is a fragmentary perspective view of the distal-end portion, schematically illustrating the manner in which a protrusive portion has a distal-end surface immersed in a liquid.

FIG. 14 is a fragmentary perspective view of the distal-end portion, illustrating the manner in which a nozzle is mounted in a suction port depicted in FIG. 13.

FIG. 15 is a fragmentary perspective view illustrating the definition of a mount area on a distal-end surface of a distal-end portion of the nozzle depicted in FIG. 14.

FIG. 16 is a plan view depicting the definition of a mount area that is different from the mount area depicted in FIG. 15 on the distal-end surface of the distal-end portion of the nozzle depicted in FIG. 14.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

Embodiments of the present disclosure will be described hereinafter with reference to the drawings. The figures are schematic and it is to be noted that the relationship between thicknesses and widths of various parts, the ratios of various parts, and so on illustrated in the figures are different from those in reality. Parts illustrated in some figures have different dimensions and ratios between those figures.

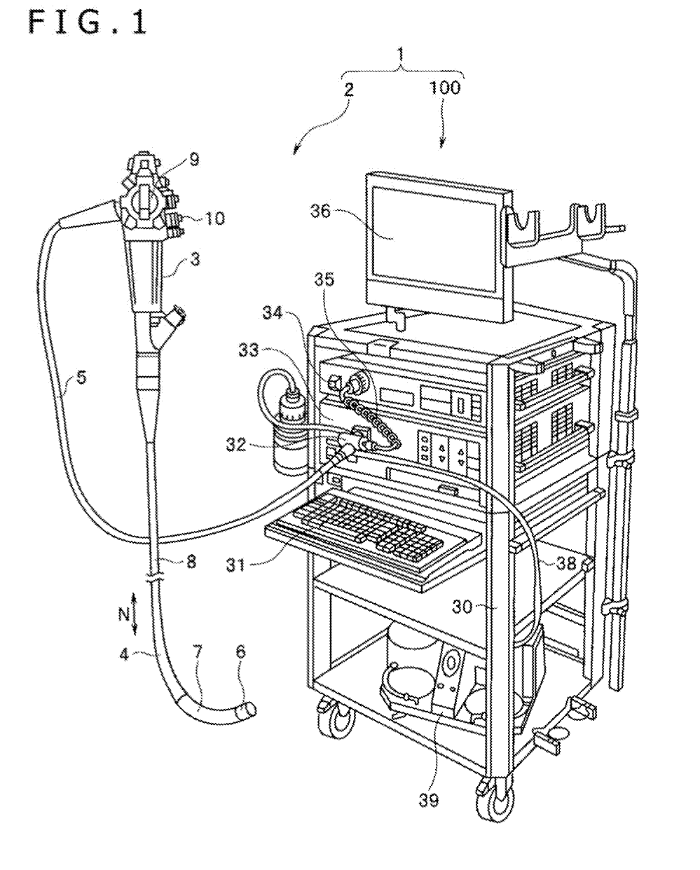

FIG. 1 is a perspective view schematically illustrating an example of an endoscopic apparatus including an endoscope and a peripheral device according to a first embodiment.

As depicted in FIG. 1, an endoscopic apparatus 1 includes an endoscope 2 and a peripheral device 100.

The endoscope 2 includes an insertion portion 4 to be inserted into a body and a manipulator 3 joined to the proximal end of the insertion portion 4 in longitudinal directions N, hereinafter simply referred to as “proximal end”.

The endoscope 2 also includes a universal cord 5 and a connector 32. The universal cord 5 extends from the manipulator 3. The connector 32 is disposed on an extended end of the universal cord 5.

The peripheral device 100 includes a keyboard 31, a light source device 33, a video processor 34, a connection cable 35 electrically interconnecting the light source device 33 and the video processor 34, a monitor 36, and a suction pump 39, all mounted on a mount base 30.

The endoscope 2 and the peripheral device 100 thus constructed are connected to each other by a connector 32.

The connector 32 is connected to the light source device 33 of the peripheral device 100, for example. The connector 32 includes a mouthpiece, not depicted, connected to the proximal end of a suction channel 57 in FIG. 5. The suction channel 57 is defined in the insertion portion 4, the manipulator 3, and the universal cord 5. A tube 38 is extending from the suction pump 39 and is connected to the mouthpiece.

The connector 32 also includes a light guide mouthpiece, electric contacts, etc. that make up the proximal end of a light guide, not depicted, inserted in the insertion portion 4, the manipulator 3, and the universal cord 5.

The manipulator 3 of the endoscope 2 has a bending manipulation knob 9, a suction button 10, and so on. The suction button 10 has an opening, not depicted, defined therein that is held in fluid communication with the suction channel 57.

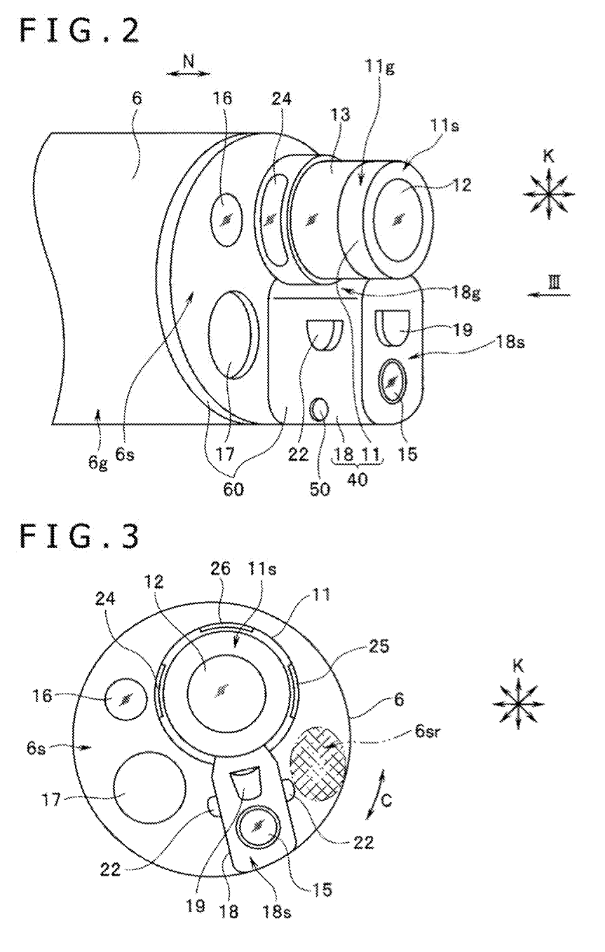

After being actuated, the suction pump 39 draws in ambient air through the opening in the suction button 10, the suction channel 57, and the tube 38. When the user closes the opening in the suction button 10, the suction pump 39 draws in a liquid W in the body through a suction port 17 in FIG. 2, the suction channel 57, and the tube 38.

The insertion portion 4 of the endoscope 2 includes a distal-end portion 6, a bendable portion 7, and a flexible tube 8. The distal-end portion 6 is positioned on the distal-end side of the insertion portion 4 in the longitudinal directions N, hereinafter simply referred to as “distal end side”. The bendable portion 7 is joined to the proximal end of the distal-end portion 6. The flexible tube 8 is joined to the proximal end of the bendable portion 7.

The bendable portion 7 is bendable in four directions, e.g., upward, downward, leftward, and rightward directions, by the bending manipulation knob 9 on the manipulator 3.

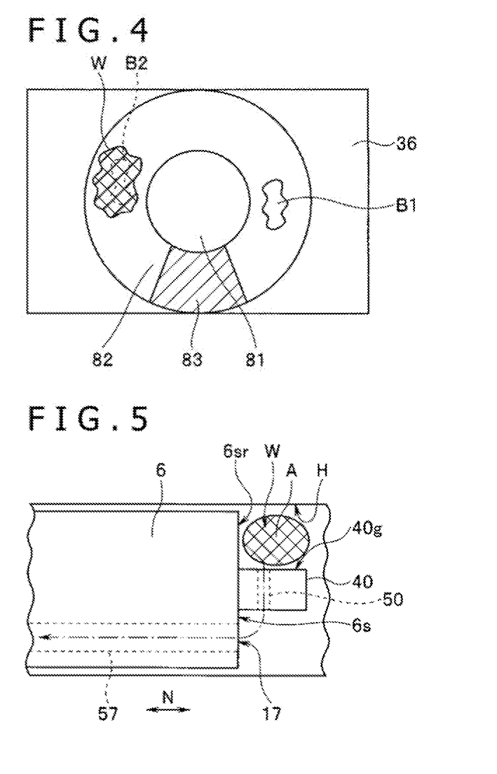

Next, structural details of the distal-end portion 6 will be described hereinafter with reference to FIGS. 2 through 5. FIG. 2 is a fragmentary perspective view illustrating at an enlarged scale the distal-end portion depicted in FIG. 1. FIG. 3 is a plan view of the distal-end portion depicted in FIG. 2, as viewed from the direction III in FIG. 2. FIG. 4 is a view illustrating a forward subject image and a side subject image displayed on the monitor depicted in FIG. 1. FIG. 5 is a view schematically illustrating the manner in which a liquid in the body is drawn from the suction port into the suction channel through a passageway depicted in FIG. 2.

As depicted in FIG. 2, a protrusive portion 40 protrudes in a columnar shape from a distal-end surface 6s of the distal-end portion 6 forwardly in the longitudinal directions N, hereinafter simply referred to as “forwardly”. The protrusive portion 40 is disposed on the distal-end surface 6s.

The protrusive portion 40 includes a first protrusive part 11 and a second protrusive part 18 disposed adjacent to the first protrusive part 11.

As depicted in FIGS. 2 and 3, the first protrusive part 11 houses therein a forward observation lens 12. The forward observation lens 12 is used as a “forward subject image acquiring section” for observing a region including a direction along the longitudinal directions N. The forward observation lens 12 acquires a forward subject image 81 in FIG. 4 and is exposed on a distal-end surface 11s of the first protrusive part 11.

The forward subject image 81 is a subject image of a subject positioned in the longitudinal directions N including a region forward of the distal-end surface 11s.

As depicted in FIG. 2, the first protrusive part 11 houses therein a circumferential observation lens 13. The circumferential observation lens 13 is used as a “side subject image acquiring section” exposed circumferentially along an outer circumferential surface 11g of the first protrusive part 11. The circumferential observation lens 13 observes a region including circumferential directions transverse to the longitudinal directions N and acquires a side subject image 82 in FIG. 4.

In the first protrusive part 11, although not depicted, the circumferential observation lens 13 is positioned rearwardly in the longitudinal directions N of the forward observation lens 12, hereinafter simply referred to as “rearwardly”.

The side subject image 82 is a subject image of a subject positioned in the circumferential directions that represent radial directions K substantially perpendicular to the longitudinal directions N. The circumferential directions will hereinafter be also denoted by K.

The distal-end portion 6 houses therein a rearward lens group, not depicted, including a plurality of lenses disposed rearwardly of the circumferential observation lens 13. An image capturing section such as a CCD or the like is disposed at the focused position of the rearward lens group.

The image capturing section captures images of a forward subject acquired by the forward observation lens 12 and a circumferential subject acquired by the circumferential observation lens 13.

(i) An arrangement for introducing light from the forward subject through the forward observation lens 12, the circumferential observation lens 13, and the rearward lens group into the image capturing section, and (ii) an arrangement for introducing light from the circumferential subject through the circumferential observation lens 13 and the rearward lens group into the image capturing section are well understood in the art, and will not be described in detail hereinafter.

The forward subject image 81 and the side subject image 82 are captured by the image capturing section and are processed, then display image signals that represent them are generated and output to the monitor 36.

As a result, as depicted in FIG. 4, the forward subject image 81 is displayed in a substantially circular shape centrally on the monitor 36, and the side subject image 82 is displayed in a substantially C shape around the outer circumference of the forward subject image 81.

As depicted in FIG. 4, the side subject image 82 is displayed in a C shape, rather than an annular shape, on the monitor 36, i.e., leaving a non-display area 83, because part of the field of vision of the circumferential observation lens 13 is blocked by the second protrusive part 18.

As depicted in FIGS. 2 and 3, three illumination lenses 24, 25, and 26 supplies illumination light in the circumferential directions K and are disposed on the outer circumferential surface 11g of the first protrusive part 11 rearwardly of the circumferential observation lens 13. Three illumination lenses 24, 25, and 26 are angularly spaced apart by approximately 90 degrees in peripheral directions C, for example, as depicted in FIG. 3. The number of the illumination lenses is not limited to three.

The second protrusive part 18 is constructed on a cover 60 covering the distal-end surface 6s of the distal-end portion 6 and a distal-end side of an outer circumferential surface 6g thereof, except for the first protrusive part 11, an illumination lens 16 to be described hereinafter, and the suction port 17.

Therefore, the second protrusive part 18 is separate from the distal-end portion 6 and the first protrusive part 11, and mounted on the distal-end portion 6. The second protrusive part 18 may alternatively be formed integral with the distal-end portion 6 and the first protrusive part 11.

A direct-viewing observation window nozzle 19 supplies a fluid to the forward observation lens 12 and is disposed on a distal-end surface 18s of the second protrusive part 18. An illumination lens 15 for supplying illumination light forwardly of the distal-end surface 18s is also disposed on the distal-end surface 18s.

Two circumferential observation window nozzles 22, for example, supplies a fluid to the circumferential observation lens 13 and are disposed on an outer circumferential surface 18g of the second protrusive part 18. The number of the circumferential observation window nozzles 22 is not limited to two.

The suction channel 57 (FIG. 5) for drawing in the liquid W in the body has a distal end in the longitudinal directions N, hereinafter simply referred to as “distal end”. The distal end of the suction channel 57 is defined as the suction port 17 in the distal-end surface 6s of the distal-end portion 6.

If the endoscope 2 is a medical endoscope, then the liquid W may be a body fluid, blood, a living tissue, dirt, or the like in the body.

An illumination lens 16 supplies illumination lens forwardly of the distal-end surface 6s and is disposed on the distal-end surface 6s.

As depicted in FIGS. 3 and 5, the second protrusive part 18 has a passageway 50 defined therein for passing therethrough the liquid W that is positioned in facing relation to the distal-end surface 6s and opposite the suction port 17 across the protrusive portion 40, toward the suction port 17.

Specifically, as depicted in FIGS. 2 and 5, the passageway 50 is defined as a hole extending through the second protrusive part 18 in directions substantially perpendicular to the longitudinal directions N, at a position in the second protrusive part 18 outside the field of vision of the circumferential observation lens 13.

The hole may have a diameter of approximately 1 mm, for example, though it is not limited to such a diameter depending on the size of the second protrusive part 18.

More specifically, as depicted in FIGS. 3 and 5, the passageway 50 is defined at a position in the second protrusive part 18 for passing therethrough the liquid W to the suction port 17 from an area “A” surrounded by (i) an outer circumferential surface 40g of the protrusive portion 40, (ii) a position 6sr on the distal-end surface 6s that is opposite the suction port 17 across the protrusive portion 40, and (iii) an outer surface H of the body.

Furthermore, in the second protrusive part 18, the passageway 50 is disposed out of physical interference with a fluid tube and a lightguide, not depicted. The fluid tube is held in fluid communication with the direct-viewing observation window nozzle 19. The lightguide is not depicted and supplies illumination light to the illumination lens 15.

According to the present embodiment, as described hereinbefore, the passageway 50 is defined in the second protrusive part 18. The liquid W is positioned in facing relation to the distal-end surface 6s and opposite the suction port 17 across the protrusive portion 40. The liquid W is passing through the passageway 50 toward the suction port 17.

With this arrangement, as depicted in FIG. 4, if the user views the liquid W together with a lesion B1 on the monitor 36 while observing the side subject image 82 using the circumferential observation lens 13, for example, the user closes the opening in the suction button 10.

The liquid W in the area “A” surrounded by (i) the outer circumferential surface 40g of the protrusive portion 40, (ii) the position 6sr on the distal-end surface 6s that is opposite the suction port 17 across the protrusive portion 40, and (iii) the outer surface H of the body is thus drawn in from the suction port 17 through the passageway 50 without being obstructed by the protrusive portion 40, as depicted in FIG. 5, while the user is viewing the lesion B1.

Therefore, as depicted in FIG. 4, even if a lesion B2 has been covered with the liquid W, since the liquid W can be drawn in, i.e., since the liquid W can be removed from the area “A”, the user is prevented from overlooking the lesion B2.

Furthermore, inasmuch as it is not necessary to change the position of the distal-end portion 6 for drawing in the liquid W, the user can draw in the liquid W without losing sight of the lesion B1.

There is thus provided the endoscope 2 incorporating the arrangement for drawing in, from the suction port 17 that is open in the distal-end surface 6s of the distal-end portion 6 of the insertion portion 4, the liquid W in the body without moving the observational field of vision. The liquid W is positioned opposite the suction port 17 across the protrusive portion 40.

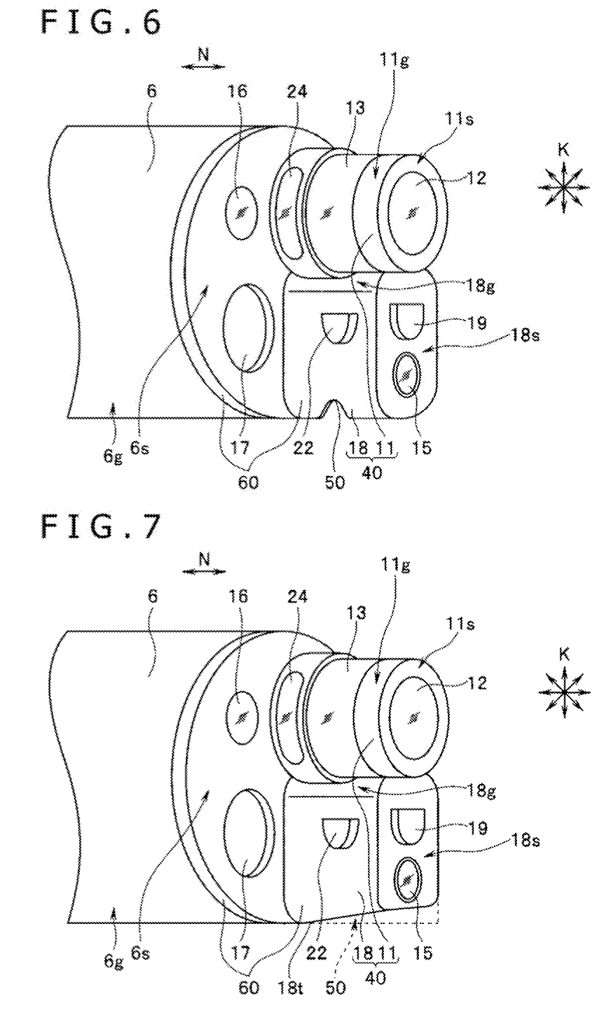

A modification will be described hereinafter with reference to FIG. 6. FIG. 6 is a fragmentary perspective view of a distal-end portion according to the modification in which the passageway depicted in FIG. 2 is defined as a groove.

In the present embodiment described hereinbefore, the passageway 50 is defined as a hole extending through the second protrusive part 18 in directions substantially perpendicular to the longitudinal directions N, at a position in the second protrusive part 18 outside the field of vision of the circumferential observation lens 13.

The passageway 50 is not limited to such a configuration, but, as depicted in FIG. 6, may be defined as a groove extending through the second protrusive part 18 in directions substantially perpendicular to the longitudinal directions N, in the bottom surface of the second protrusive part 18 at a position in the second protrusive part 18 outside the field of vision of the circumferential observation lens 13. This arrangement also offers the same advantages as those of the present embodiment described hereinbefore.

A modification will be described hereinafter with reference to FIG. 7. FIG. 7 is a fragmentary perspective view of a distal-end portion according to a modification in which the passageway depicted in FIG. 2 is defined as a space created by a tapered surface on the second protrusive part.

As depicted in FIG. 7, the passageway 50 may be defined as a space created by a tapered surface 18t extending through the second protrusive part 18 in directions substantially perpendicular to the longitudinal directions N and inclined to make the second protrusive part 18 tapering off forwardly, on the bottom surface of the second protrusive part 18 at a position in the second protrusive part 18 outside the field of vision of the circumferential observation lens 13. This arrangement also offers the same advantages as those of the present embodiment described hereinbefore.

Moreover, the passageway 50 may be hydrophilized or finely grooved in its inlet close to the area A (see FIG. 5) for attracting the liquid W into the passageway 50.

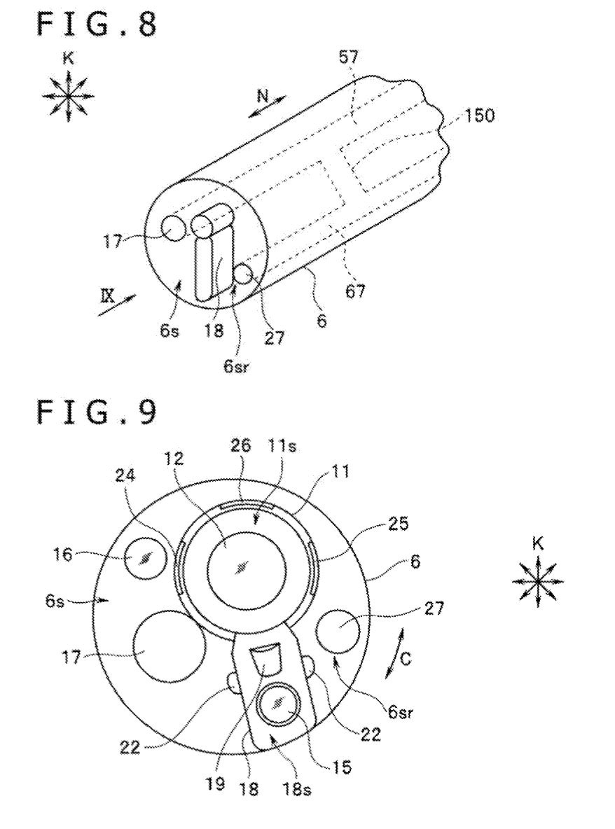

FIG. 8 is a fragmentary perspective view schematically illustrating at an enlarged scale a distal-end portion of an insertion portion of an endoscope according to a second embodiment. FIG. 9 is a plan view of the distal-end portion depicted in FIG. 8, as viewed from the direction IX in FIG. 8.

The endoscope according to the second embodiment is structurally different from the endoscope according to the first embodiment depicted in FIGS. 1 through 7 described hereinbefore in that a forward water delivery channel is connected to the suction channel to draw in the liquid W that is positioned opposite the suction port that is open at the distal-end surface through an opening of the forward water delivery channel.

Therefore, only the differences will be described hereinafter, and those parts which are identical to those according to the first embodiment are denoted by identical reference characters and will not be described hereinafter.

According to the present embodiment, as depicted in FIGS. 8 and 9, an opening 27 of a forward water delivery channel 67 extending through the insertion portion 4, the manipulator 3, the universal cord 5, and the connector 32 is defined in the distal-end surface 6s at a position 6sr that is opposite the suction port 17 across the protrusive portion 40.

The forward water delivery channel 67 primarily serves to supply a fluid forwardly in the longitudinal directions N through the opening 27.

The forward water delivery channel 67 is connected to the suction channel 57 through a connection channel 150 in the insertion portion 4. Other structural details are identical to those according to the first embodiment.

With this arrangement, while viewing the lesion B1, the user can draw in the liquid W that faces the position 6sr that is opposite the suction port 17 across the protrusive portion 40 on the distal-end surface 6s, i.e., the liquid W (see FIG. 5) in the area A surrounded by the outer circumferential surface 40g of the protrusive portion 40, the position 6sr, and the outer surface H of the body, from the opening 27 through the forward water delivery channel 67 connected to the suction channel 57 through the connection channel 150 without being obstructed by the protrusive portion 40, as depicted in FIG. 5. Consequently, this arrangement offers the same advantages as those of the first embodiment described hereinbefore.

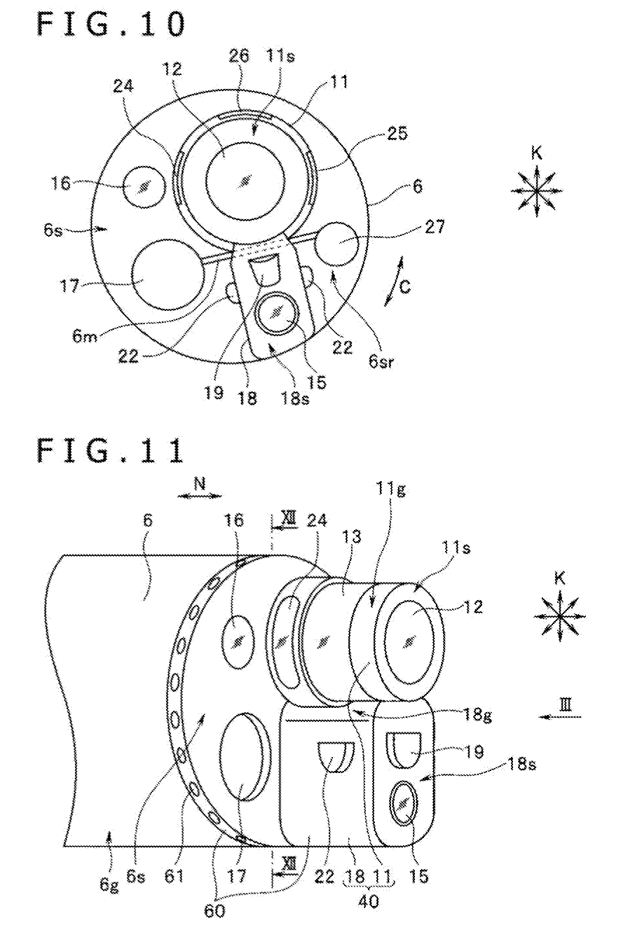

A modification will be described hereinafter with reference to FIG. 10. FIG. 10 is a plan view illustrating a modification in which the distal-end portion depicted in FIG. 8 has a groove defined in a distal-end surface thereof and providing fluid communication between the suction port and the opening of the forward water delivery channel.

As depicted in FIG. 10, the forward water delivery channel 67 and the suction channel 57 may be connected to each other by a groove 6m that provides fluid communication between the suction port 17 and the opening 27 which are defined in the distal-end surface 6s.

With this arrangement, the liquid W that faces the position 6sr that is opposite the suction port 17 across the protrusive portion 40 on the distal-end surface 6s is drawn in through the groove 6m from the suction port 17. Consequently, this arrangement offers the same advantages as those of the present embodiment described hereinbefore.

The groove is not limited to one defined in the distal-end surface 6s, but may be defined in the cover 60 as the second protrusive part 18.

In this case, as with the passageway 50 according to the first embodiment described hereinbefore, the groove should be disposed, to the cover 60, out of physical interference with a fluid tube, not depicted, held in fluid communication with the direct-viewing observation window nozzle 19 and a lightguide, not depicted, for supplying illumination light to the illumination lens 15.

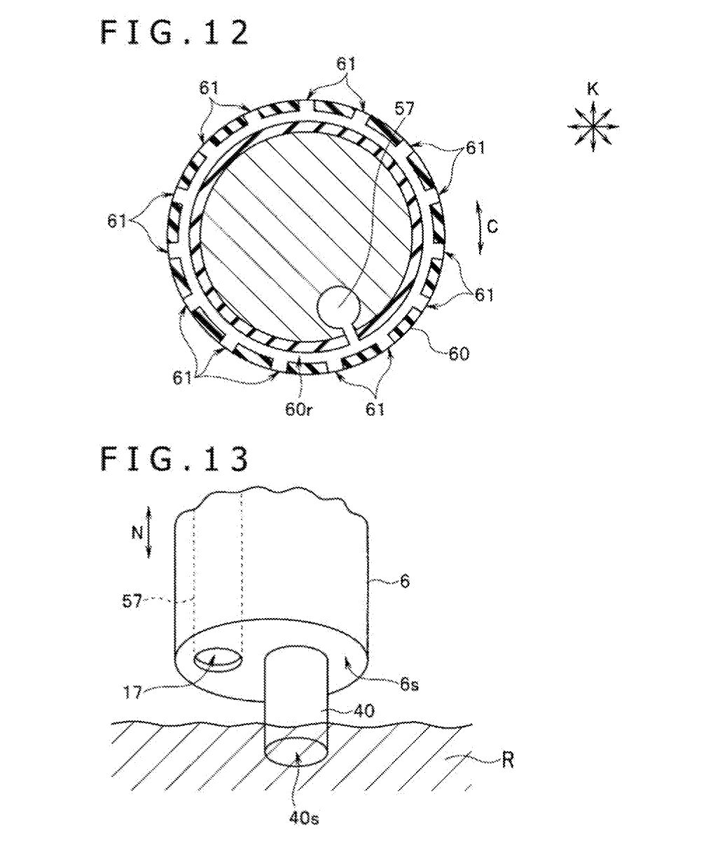

Another modification will be described hereinafter with reference to FIGS. 11 and 12. FIG. 11 is a fragmentary perspective view illustrating a modification in which a plurality of suction holes are defined in a cover that covers an outer circumferential surface of the distal-end portion depicted in FIG. 8. FIG. 12 is a cross-sectional view of a distal-end portion and a cover, taken along line XII-XII of FIG. 11.

As depicted in FIGS. 11 and 12, the cover 60 that covers the distal-end portion 6 may have a plurality of suction holes 61 defined in a section thereof that covers the distal-end side of the outer circumferential surface 6g of the distal-end portion 6, the suction holes 61 being connected to the suction channel 57 through a tubular passage 60r defined in the peripheral directions C in an inner circumferential surface of the cover 60.

With this arrangement, the liquid W in the area A is drawn into the suction channel 57 through the suction holes 61 and the tubular passage 60r. Consequently, this arrangement offers the same advantages as those of the present embodiment described hereinbefore.

In the present embodiment described hereinbefore, the opening 27 of the forward water delivery channel 67 is defined at the position 6sr, and the forward water delivery channel 67 is connected to the suction channel 57 through the connection channel 150.

Such a structure is not restrictive. A suction port of another suction channel, not depicted, extending through the insertion portion 4, the manipulator 3, the universal cord 5, and the connector 32 and connected to the suction pump 39 may be defined at the position 6sr, and the liquid W in the area A (see FIG. 5) can be drawn in from the suction port of the other suction tubular passage. Consequently, this arrangement offers the same advantages as those of the present embodiment described hereinbefore.

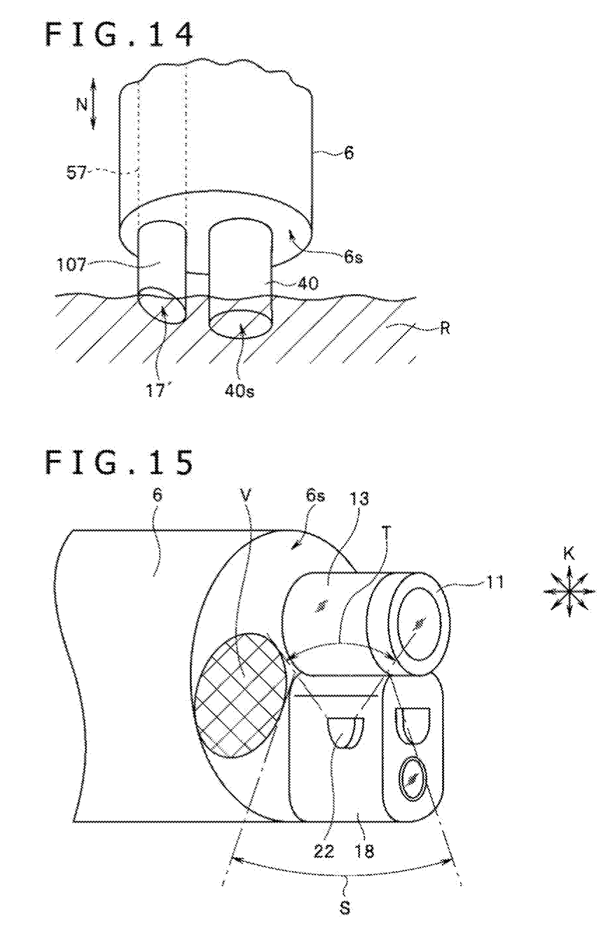

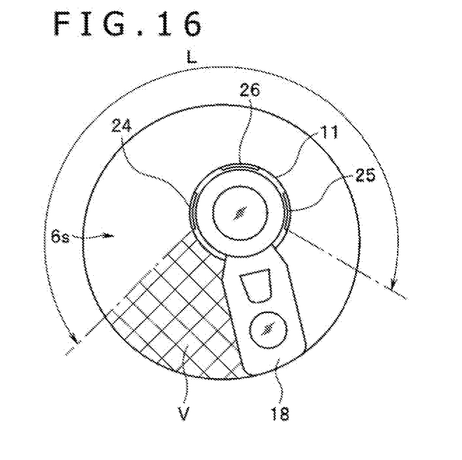

FIG. 13 is a fragmentary perspective view of the distal-end portion, schematically illustrating the manner in which a protrusive portion has a distal-end surface immersed in a liquid. FIG. 14 is a fragmentary perspective view of the distal-end portion, illustrating the manner in which a nozzle is mounted in a suction port depicted in FIG. 13. FIG. 15 is a fragmentary perspective view illustrating the definition of a mount area on a distal-end surface of a distal-end portion of the nozzle depicted in FIG. 14. FIG. 16 is a plan view depicting the definition of a mount area that is different from the mount area depicted in FIG. 15 on the distal-end surface of the distal-end portion of the nozzle depicted in FIG. 14.

As described hereinbefore, since the suction port 17 is open at the distal-end surface 6s, a liquid R cannot be drawn in from the suction port 17 when the protrusive portion 40 has a distal-end surface 40s immersed in the liquid R, as depicted in FIG. 13.

As depicted in FIG. 14, a nozzle 107 having a suction port 17′ that is capable of contacting the liquid R when the distal-end surface 40s is immersed in the liquid R may be mounted in the suction port 17.

In this case, however, the nozzle 107 needs to be disposed in an area V that does not obstruct a fluid supply range T of the circumferential observation window nozzle 22 and a field of vision S of the circumferential observation lens 13, as depicted in FIG. 15, and also in an area V that does not obstruct an illumination range L of the illumination lenses 24 through 26, as depicted in FIG. 16.

In order to meet these conditions, the nozzle 107 may have a distal-end side of a slanted shape, as depicted in FIG. 14.

In the first and second embodiments described hereinbefore, one monitor displays a plurality of subject images by way of example. This arrangement is not restrictive, and subject images may be displayed on a plurality of monitors.

The endoscope 2 according to the first and second embodiments described hereinbefore is not limited to medical use, but is also applicable to industrial use.

In sum, one aspect of the disclosed technology is directed to an endoscope comprises an insertion portion having a distal-end surface configured to be inserted into a body. The insertion portion includes a suction port being formed in the distal-end surface for drawing in liquid from the body. A protrusive portion having a passageway formed thereto. The protrusive portion is protruded from the distal-end surface. The passageway is formed in the protrusive portion to remove the liquid positioned in facing relationship with respect to the distal-end surface and opposite of the suction port across the protrusive portion toward the suction port.

The protrusive portion includes a first protrusive part having a forward subject image acquiring section for observing a region including a direction along longitudinal directions of the insertion portion. A second protrusive part configured to be disposed adjacent to the first protrusive part in which the passageway is formed in the second protrusive part. A side subject image acquiring section disposed on an outer circumferential surface of the first protrusive part for observing a region including a direction transverse to the longitudinal directions. The passageway is disposed in a position in the second protrusive part outside of the field of vision of the side subject image acquiring section. The passageway extends through the second protrusive part in directions substantially perpendicular to the longitudinal directions. The second protrusive part includes a groove formed in a bottom surface thereof in directions substantially perpendicular to the longitudinal directions. The second protrusive part includes a bottom surface being tapered off forwardly in the longitudinal directions. The passageway is disposed in a position in the protrusive part for removing the liquid in a region surrounded by an outer circumferential surface of the protrusive portion wherein the position is opposite the suction port in the distal-end surface across the protrusive part.

Another aspect of the disclosed technology is directed to an endoscope comprises an insertion portion having a distal-end surface configured to be inserted into a body. The insertion portion includes a suction port being formed in the distal-end surface for removing liquid from the body. A protrusive portion is protruded from the distal-end surface. A liquid removing means is formed on the protrusive portion so as to remove the liquid at a location facing the distal-end surface and is opposite from the suction port location with respect to the protrusive portion. The liquid removing means comprises a passageway formed in the protrusive portion. The protrusive portion is defined by respective first and second protrusive parts each of which is protruded from the distal-end surface and the passageway is formed in the second protrusive part. The liquid removing means comprises a groove formed in a bottom surface of the second protrusive part. The liquid removing means comprises a tapered surface being formed on the protrusive portion.

A further aspect of the disclosed technology is directed to an endoscope comprises an insertion portion having a suction port formed on a distal-end surface to remove liquid from a body. Respective first and second protrusive parts each of which is protruded from the distal-end surface. A liquid removing means is formed on the second protrusive part so as to remove the liquid at a location facing the distal-end surface and is opposite from the suction port location with respect to the first and second protrusive parts. The liquid removing means comprises a passageway formed on the second protrusive part. The liquid removing means comprises a groove formed in a bottom surface of the second protrusive part. The liquid removing means comprises a tapered surface formed on the second protrusive part. The first protrusive part includes a forward subject image acquiring section for observing a region including a direction along longitudinal directions of the insertion portion and the second protrusive part is configured to be disposed adjacent to the first protrusive part. The endoscope further comprises a side subject image acquiring section disposed on an outer circumferential surface of the first protrusive part for observing a region including a direction transverse to the longitudinal directions. While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. An endoscope comprising:
an insertion portion having a distal-end surface configured to be inserted into a body, the insertion portion includes:
a suction port being formed in the distal-end surface for drawing in liquid from the body; and
a protrusive portion having a passageway formed thereto, the protrusive portion being protruded from the distal-end surface wherein the passageway being formed in the protrusive portion to remove the liquid positioned in facing relationship with respect to the distal-end surface and opposite of the suction port across the protrusive portion toward the suction port.

2. The endoscope of claim 1,
wherein the protrusive portion includes:
a first protrusive part having a forward subject image acquiring section for observing a region including a direction along longitudinal directions of the insertion portion;
a second protrusive part configured to be disposed adjacent to the first protrusive part, and
wherein the passageway is formed in the second protrusive part.

3. The endoscope of claim 2 further comprising:
a side subject image acquiring section disposed on an outer circumferential surface of the first protrusive part for observing a region including a direction transverse to the longitudinal directions.

4. The endoscope of claim 3, wherein the passageway is disposed in a position in the second protrusive part outside of the field of vision of the side subject image acquiring section.

5. The endoscope of claim 4, wherein the passageway extends through the second protrusive part in directions substantially perpendicular to the longitudinal directions.

6. The endoscope of claim 4, wherein the second protrusive part includes a groove formed in a bottom surface thereof in directions substantially perpendicular to the longitudinal directions.

7. The endoscope of claim 4, wherein the second protrusive part includes a bottom surface being tapered off forwardly in the longitudinal directions.

8. The endoscope of claim 1, wherein the passageway is disposed in a position in the protrusive part for removing the liquid in a region surrounded by an outer circumferential surface of the protrusive portion wherein the position being opposite the suction port in the distal-end surface across the protrusive part.

9. An endoscope comprising:
an insertion portion having a distal-end surface configured to be inserted into a body, the insertion portion includes:
a suction port being formed in the distal-end surface for removing liquid from the body;
a protrusive portion being protruded from the distal-end surface; and
a liquid removing means being formed on the protrusive portion so as to remove the liquid at a location facing the distal-end surface and being opposite from the suction port location with respect to the protrusive portion.

10. The endoscope of claim 9, wherein the liquid removing means comprises a passageway formed in the protrusive portion.

11. The endoscope of claim 10, wherein the protrusive portion is defined by respective first and second protrusive parts each of which being protruded from the distal-end surface and wherein the passageway is formed in the second protrusive part.

12. The endoscope of claim 9, wherein the liquid removing means comprises a groove formed in a bottom surface of the second protrusive part.

13. The endoscope of claim 9, wherein the liquid removing means comprises a tapered surface being formed on the protrusive portion.

14. An endoscope comprising:
an insertion portion having a suction port formed on a distal-end surface to remove liquid from a body,
respective first and second protrusive parts each of which being protruded from the distal-end surface, and
a liquid removing means being formed on the second protrusive part so as to remove the liquid at a location facing the distal-end surface and being opposite from the suction port location with respect to the first and second protrusive parts.

15. The endoscope of claim 14, wherein the first protrusive part includes a forward subject image acquiring section for observing a region including a direction along longitudinal directions of the insertion portion and the second protrusive part is configured to be disposed adjacent to the first protrusive part.

16. The endoscope of claim 15 further comprising a side subject image acquiring section disposed on an outer circumferential surface of the first protrusive part for observing a region including a direction transverse to the longitudinal directions.

17. The endoscope of claim 14, wherein the liquid removing means comprises a passageway formed on the second protrusive part.

18. The endoscope of claim 14, wherein the liquid removing means comprises a groove formed in a bottom surface of the second protrusive part.

19. The endoscope of claim 14, wherein the liquid removing means comprises a tapered surface being formed on the second protrusive part.
